(19) United States
(12) Patent Application Publication
(10) Pub. No.: US 2023/0293636 A1
(43) Pub. Date: Sep. 21, 2023

(54) PHARMACEUTICAL COMPOSITION FOR THE PREVENTION OR TREATMENT OF POST-SURGICAL PAIN

(71) Applicant: PEPTINOV, PARIS (FR)

(72) Inventors: Rene AZOULAI, PARIS (FR); Xavier CASSARD, TOULOUSE (FR); Jean-Pierre SALLES, EGUILLES (FR); Jean-Francois ZAGURY, LE VESINET (FR)

(21) Appl. No.: 17/758,281

(22) PCT Filed: Dec. 31, 2020
(86) PCT No.: PCT/EP2020/088091

§ 371 Date: Dec. 21, 2022

(30) Foreign Application Priority Data
Dec. 31, 2019 (EP) 19306800.4

(51) Int. Cl. A61K 38/20; C07K 16/24; A61P 29/02

(52) U.S. Cl. CPC A61K 38/204; A61P 29/02; C07K 16/248; A61K 2039/505

(57) ABSTRACT

The present invention relates to an anti-IL-6 agent for use in a method of preventing or treating post-operative pain in an individual.

### FIELD OF THE INVENTION

The present invention relates to a pharmaceutical composition useful for the prevention or treatment of post-operative pain. in particular persistent or chronic post-operative pain.

### TECHNICAL BACKGROUND

Surgical interventions are very often followed by immediate and medium-term post-operative pain, even chronic pain. For some operations, chronic pain can persist for more than a year, with rates exceeding 20% for knee arthroplasties or even 40% for cardiac surgeries. The persistence of this pain and its intensity can significantly impact the quality of life of patients and also their professional activity, and this poses a major public health problem.

Several approaches have been explored to decrease immediate post-operative pain, but very few studies have focused on chronic post-operative pain despite its significant morbidity.

For many operations, such as knee arthroplasty for example, a significant proportion of patients have been shown to have persistent pain for several months and sometimes several years, often associated with a consequent reduction in the quality of life of the patients.

Few approaches have been tested to prevent this type of prolonged pain. Aside from recognized risk factors such as depression or pre-operative pain intensity, it has been described that chronic pain may in part result from inadequate treatment of immediate post-operative pain (Pogatzki-Zahn et al. (2017) Pain Rep. 2:e588: Searle & Simpson (2010) Continuing Education in Anaesthesia Critical Care & Pain 10:12-14).

It is therefore necessary to identify useful agents for the management of post-operative pain, in particular chronic post-operative pain.

### SUMMARY OF THE INVENTION

The present invention arises from the unexpected finding by the inventors that, in the murine cutaneous-muscular incision model, post-operative pain was significantly reduced by blocking IL-6 either by administration of anti-IL-6 antibodies or by administration of active anti-lL-6 immunotherapy.

Thus, the present invention relates to an anti-lL-6 agent for use in a method of preventing or treating post-operative, in particular chronic, pain in an individual.

The present invention also relates to a method of preventing or treating post-operative, in particular chronic, pain in an individual, wherein the individual is administered a prophylactically or therapeutically effective amount of an anti-lL-6 agent.

The present invention also relates to the use of an anti-IL-6 agent for the preparation of a medicament, vaccine or pharmaceutical composition for the prevention or treatment of post-operative, in particular chronic, pain in an individual.

In a preferred embodiment of the invention, the anti-IL-6 agent is combined with at least one other anti-lL-6 agent.

### DESCRIPTION OF THE INVENTION

As a preliminary remark, it should be noted that the term “comprising” means “including”, “containing” or “encompassing”, i.e. when an object “comprises” an element or several elements, elements other than those mentioned may also be included in the object. Conversely, the expression “consisting or means “constituted of”. i.e. when an object “consists of” an element or elements, the object cannot include other elements than those mentioned.

### Post-Operative Pain

As understood herein, post-operative pain according to the invention is pain following a surgical operation.

Preferably, the post-operative pain according to the invention is not immediate, i.e. it does not appear as soon as the operation is over, in particular as soon as the individual who underwent the operation wakes up if the individual was put to sleep for the operation.

Preferably, the post-operative pain according to the invention is not acute.

Preferably, the post-operative pain according to the invention is chronic pain.

Preferably, the post-operative pain according to the invention is persistent pain, in particular pain that persists for more than 1 week, 2 weeks, 3 weeks, 4 weeks, 2 months, 3 months. 6 months or 1 year after the operation.

Preferably, the post-operative pain according to the invention is following an arthroplasty, especially of the knee or hip, a thoracic operation, especially a cardiac operation, a thoracotomy or sternotomy, a breast operation, a hernia operation, a hysterectomy, a cholecystectomy, a knee arthroscopy, or a caesarean section.

### Agent

Interleukin 6 (IL-6), also sometimes referred to as B-cell stimulatory factor 2 (BSF-2), cytotoxic T-cell differentiation factor (CDF), hybridoma growth factor, or interferon β-2 (IFN-β-2), is well known to the skilled person. Numerous sequences of IL-6 from various animal species are available in sequence databases. As an example, a human IL-6 is described in the UniProt/Swissprot database as P05231 (SEQ lD NO: 1). As an example also, the alpha subunit of the human IL-6 receptor is described in the UniProt/Swissprot database under the reference P08887 (SEQ lD NO: 4).

As intended herein, an anti-IL-6 agent is an agent capable of blocking, in part or completely, the action of IL-6 in vivo or in the body.

The anti-IL-6 agent according to the invention may be of any type. In particular, it may be a protein, a nucleic acid or a small molecule.

Preferably, the anti-lL-6 agent according to the invention is specifically directed against IL-6 or against the IL-6 receptor in order to block the IL-6 pathway. Preferably, the anti-lL-6 agent according to the invention is an immunotherapeutic agent.

More preferably, the anti-IL-6 agent according to the invention is a passive immunotherapy agent. Even more preferably, the anti-IL-6 agent according to the invention is an anti-IL-6 antibody, in particular a monoclonal antibody, or an IL-6-binding portion of an antibody.

More preferably also, the agent is selected from the group consisting of an IL-6 receptor or an IL-6 binding portion thereof or an anti-IL-6 aptamer.

Anti-IL-6 agents, in particular antibodies, antibody fragments, receptors, receptor parts, and aptamers, according to the invention, are said to be specifically directed against IL-6 when they do not substantially exhibit binding to another polypeptide. which does not comprise IL-6, under conditions which allow binding of the antibodies, antibody fragment, and aptamers according to the invention to IL-6.

The antibody according to the invention may be polyclonal or monoclonal, preferably monoclonal. Examples of monoclonal anti-IL-6 antibodies according to the invention include toclizumab or clazakizumab. Furthermore, as understood herein, the “antibody fragments” comprise at least one antigen-binding portion of the antibody from which they are derived, and are in particular of the Fab. Fab′, F(ab′)₂. disulfide-stabilized Fv (dsFv), dimerized (diabody), trimerized, tetramerized or pentamerized V region, single-chain Fv (scFv), and complementarity-determining region (CDR) types. The antibodies can be of any species, including human, mouse, rat, rabbit or camelid. Furthermore, when they are not human, they can also be humanized, i.e. the constant parts of these antibodies are partially or completely replaced by corresponding human constant parts.

The antibodies according to the invention can be obtained by immunizing an animal with a polypeptide according to the invention according to techniques well known to the person skilled in the art.

As understood herein, the aptamers are nucleic acids, in particular RNAs, capable of specifically binding to a molecular target, such as a protein. In particular, the aptamers can be obtained by implementing the SELEX technique well known to the skilled person, from the polypeptides according to the invention.

Alternatively, the anti-IL-6 agent according to the invention is more preferably an active immunotherapy agent. Still more preferably, the anti-IL-6 agent comprises, or consists of, a polypeptide derived from IL-6 or the IL-6 receptor (IL-6R), optionally bound to a carrier protein. Preferably, the polypeptide comprises, or consists of, a sequence of at least 6 amino acid residues of IL-6 or IL-6R or a variant sequence having at least 90% identity therewith.

In particular, the polypeptide according to the invention may be as described in International Application WO2013/021284, which is incorporated herein by reference. Preferably, the polypeptide according to the invention comprises at least the sequence ALAENNL (SEQ lD NO: 3) or a sequence having at least 90% sequence identity with SEQ lD NO: 3.

More preferably, the polypeptide according to the invention comprises or consists of CMNNDDALAENNLKLPECY (SEQ ID NO: 2). CESSKEALAENNLNLPKC (SEQ ID NO: 5). CESSKEALAENNLNLPKCY (SEQ ID NO: 6), or a sequence having at least 90% sequence identity with SEQ ID NO: 2, 5 or 6.

Preferably, the variant sequence according to the invention has at least 95% or 98% identity with the sequence according to the invention.

As intended herein, the percentage of identity between two peptide sequences can be determined by performing an optimal alignment over the entire length of the sequences, determining the number of aligned positions for which the amino acids are identical in each sequence and dividing this number by the total number of amino acids in the longer of the two sequences. The optimal alignment is the one that gives the highest percentage of identity between the two sequences.

The variant sequence according to the invention is such that a polypeptide consisting of the variant sequence must make it possible to elicit an immune response directed against IL-6: i.e. the administration of such a polypeptide, optionally cyclized by formation of at least one inter-cysteine disulfide bond, if necessary after addition of one or two cysteines within the polypeptide, and/or at its N-terminus and/or at its C-terminus, the polypeptide being optionally linked to a carrier molecule, in particular a carrier protein, such as KLH (Keyhole Limpet Hemocyanin), to an animal, such as a mouse, rat or rabbit, causes the production of antibodies directed against IL-6. It is well known to the skilled person how to determine whether an antibody is directed against IL-6, in particular by implementing an ELISA test. Preferably, the antibodies elicited by administration of the polypeptide are blocking or neutralizing, i.e. they prevent IL-6 from exerting all or part, in particular at least 10%, 25%, 50%, 75%, of its activity, for example measured in vitro.

The polypeptide according to the invention preferably comprises at most 200, 150, 100, 90, 80. 70, 60, 50, 40 or 30 amino acid residues. Alternatively, the polypeptide may be IL-6.

As will be apparent to the skilled person, the polypeptide according to the invention may comprise several repeats, for example 2, 3, 4. 5. 10 or 20 repeats, respectively of the sequence or variant sequence according to the invention.

Furthermore, the polypeptide according to the invention may also comprise additional sequences not derived from IL-6.

These additional sequences may, in particular, provide physicochemical characteristics allowing an improved structural presentation or an improved solubility of the polypeptide according to the invention compared to a similar polypeptide which would not comprise these additional sequences.

The additional sequences may also comprise one or more peptide linker sequences useful for inking in particular to a carrier molecule. Such peptide linker sequences typically comprise from 1 to 10, especially from 4 to 6, amino acid residues.

Furthermore, these non-IL-6 sequences may also include epitopes belonging to other proteins, allowing to elicit or generate an immune response directed against these other proteins.

In addition, the polypeptide according to the invention may comprise exogenous, preferably universal, T epitope(s) sequences, thereby enhancing the immunogenicity of the polypeptide according to the invention.

The polypeptide according to the invention may also comprise at least one sequence of a carrier protein, for example a virus-like particle (VLP), as described in particular in international application WO 05/117983 for TNF.

The polypeptide according to the invention may be in linear form or in cyclized form. Preferably, the polypeptide according to the invention is in cyclized form. This cyclization may be of any type known to the person skilled in the art.

In particular, the choice of cyclization strategy according to the invention may take into account the best antigenic presentation of the epitopes contained in the polypeptide according to the invention, and may involve only a portion of the polypeptide (cyclization within the sequence). Thus, as intended herein, when the polypeptide according to the invention is in cyclized form, only a portion of the polypeptide may be included in a cycle while the rest of the polypeptide is in linear form.

Depending on the functional groups present in the polypeptide, this cyclization can take place in several different ways, such as: from its C-terminal end to its N-terminal end, from its N-terminal end to a side chain, from a side chain to its C-terminal end or between two side chains. Among the various modalities of cyclization of polypeptides, it is possible to mention lactamization, lactonization or disulfide bond formation. In particular, when an inter-cysteine disulfide bond is formed, i.e. between the -SH radicals of two cysteines, the cysteines may already be present in the variant sequence according to the invention or in the first, second, third, fourth and fifth sequences according to the invention, or they may be added within these sequences, as well as at their N-terminal and/or C-terminal end.

In addition, the polypeptide according to the invention may comprise post-translational modifications, such as glycosylations, methylations, acylations, especially by fatty acids, or phosphorylations. In particular, the N-terminus of the polypeptide according to the invention may be acetylated and the C-terminus may be modified by amidation.

The polypeptide according to the invention may also comprise one or more analogues or derivatives of amino acids, including non-natural or non-standard amino acids, in particular norleucine (Nle).

Also preferably, the polypeptide according to the invention is attached or linked, in particular by covalent bonding, to a carrier protein.

In particular, the carrier molecule can be Keyhole Limpet Hemocyanin (KLH), Hepatitis B surface antigen (HBsAg), bovine serum albumin (BSA), tetanus toxoid (TT) and diphtheria toxoid (DT).

The diphtheria toxoid (DT) according to the invention is preferably selected from the group consisting of CRM 197, CRM 176, CRM 228, CRM 45, CRM 9, CRM 102, CRM 103, and CRM 107.

In a particularly preferred way, the carrier molecule is CRM 197.

The binding of the polypeptide according to the invention to a carrier molecule, in particular a carrier protein, can be achieved by means of a heterobifunctional coupling agent, such as N-γ-maleimidobutyryl-oxysuccinimide ester (GMBS) and the sulfo-GMBS derivative, m-maleimidobenzoyl-n-hydroxysuccinimide ester (MBS) and the sulfo-MBS derivative, succinimidyl 4-(N-maleimidomethyl)cyclohexane-1-carboxylate (SMCC), a carbodiimide, bisdiazonium-benzidine (BDB) or glutaraldehyde

When GMBS, MBS or SMCC are used, they are preferably attached to a cysteine (C). which, if it is not present in the sequence, or the variant sequence according to the invention, can be added, especially at its N-terminal or C-terminal end. Furthermore, when a cysteine is present in the sequence according to the invention at an undesired position, it is possible to implement, instead, a variant sequence in which the cysteine is substituted by another amino acid, such as a serine.

When the BDB is used, it is preferably attached to a tyrosine (Y), which, if it is not present in the sequence or variant sequence according to the invention, can be added, in particular at its N-terminal or C-terminal end. Furthermore, when a tyrosine is present in the sequence according to the invention at an undesired position, it is possible to implement, instead, a variant sequence in which the tyrosine is substituted by another amino acid, such as a phenylalanine (F).

Furthermore, the binding of the polypeptide according to the invention to a carrier molecule, in particular a carrier protein, can also be achieved by means of a peptide linker, which bind to the polypeptide according to the invention on one side and to the carrier molecule on the other side, optionally via a heterobifunctional coupling agent as defined above. Such peptide linkers typically comprise from 1 to 10, especially from 4 to 6, amino acid residues.

### Individual

Preferably, the individual according to the invention is a mammal, including a human, dog, cat or horse.

When the individual according to the invention is a human, it can be a man, a woman, or a child.

Preferably, the individual according to the invention is a human over the age of 40, 50, 60 or 70.

As will be readily understood, the individual according to the invention has undergone, or is scheduled to undergo, surgery, particularly one that is likely to produce post-operative pain according to the invention.

### Method

Preferably, the method of preventing or treating post-operative pain according to the invention is a passive immunotherapy method, including serotherapy, or an active immunotherapy method, including vaccinotherapy.

Alternatively, the method according to the invention is a method of administering a small molecule targeting the IL-6 pathway at regular intervals, including every day, every other day, or every third day.

### Administration

The anti-IL-6 agent according to the invention is preferably administered or in a form that can be administered orally, mucosally, including sublingually, parenterally, intraperitoneally, transcutaneously, intradermally, subcutaneously, intramuscularly, intravenously or intra-arterially.

The anti-lL-6 agent according to the invention is preferably in an oral galenic form, in particular in the form of a tablet, capsule, syrup, powder to be dissolved, drinkable solution or suspension; injectable, in particular in the form of a solution in ampoule, syringe or pen, of a powder to be dissolved, of a solution for slow infusion: dermal, in particular in the form of an ointment, cream, gel, solution, powder to be dissolved or patch: inhaled, in particular in the form of an aerosol; or rectal, in particular in the form of a suppository.

When the anti-IL-6 agent is a polypeptide according to the invention it can be administered in doses ranging from, for example, 1 ng to 1 g, preferably from 1 µg to 1 mg.

When the anti-IL-6 agent is an antibody, especially a monoclonal one, the amounts administered may be in the range of 0.5 to 30 mg/kg.

When the agent is a small molecule, it can be administered in doses ranging from, for example, 50 µg to 5 g per administration.

The anti-IL-6 agent may be combined with at least one pharmaceutically acceptable vehicle in a drug or pharmaceutical or vaccine composition. As used herein, a “pharmaceutically acceptable vehicle” encompasses all compounds, including excipients, that can be administered to an individual in conjunction with a pharmacological active ingredient.

Furthermore, in particular when used in a vaccine or prophylactic setting, the polypeptide according to the invention may be associated or combined with an adjuvant, or the pharmaceutical or vaccine composition, or the drug according to the invention may comprise an adjuvant. The adjuvant may be of any type adapted to increase the immune response of an individual, animal or human, to the administration of a polypeptide. Thus, it may be complete incomplete Freund’s adjuvant. Montanide ISA 51 VG, aluminum hydroxide, aluminum phosphate or calcium phosphate for example: Montanide ISA 51 VG and aluminum hydroxides or aluminum phosphate being preferred. The adjuvant can be combined with the polypeptide according to the invention by making a 1:1 mixture by volume of an adjuvant solution and a solution comprising the polypeptide.

The individual may receive the anti-IL-6 agent prior to surgery or immediately after. This administration can typically be done by regular injections or tablets, until the individual is pain free, as early as two months prior to surgery, preferably less than one month prior to surgery, possibly a few days prior to surgery, possibly after surgery but less than 3 months after surgery. The interval between administrations will depend on the pharmacology of the product. For example, if it is an antibody, in particular a monoclonal antibody, the administration can be done every 15 days or every month until the post-operative pain stops, starting, for example, in the month before the operation. If it is a small molecule, the pharmaceutical composition or the drug comprising it, for example in tablet form, will normally be taken more frequently as is often the case for small pharmaceutical molecules and may begin a few days before or after the operation, in particular from 15 days before to 15 days after the operation. In the case of active immunization, it would be preferable for immunization to begin several weeks prior to surgery so that antibodies can be generated and effective as soon as surgery is performed. Typically, the individual could be immunized 3 months prior to surgery, with booster shots every 3 weeks, until a satisfactory antibody level has been induced. There is nothing to prevent immunization closer to the operation or even after the operation.

### Combination

As used herein, the expression “in combination” or “combined” means that the anti-IL-6 agent and the other anti-IL-6 agent as defined above may be combined in the same pharmaceutical composition or drug and thus administered together, or they may be administered separately i.e. by separate routes of administration and/or separate regimens of administration, provided that when administered separately the periods of prophylactic or therapeutic activity of the anti-IL-6 agent and the other anti-IL-6 agent as defined above overlap completely or in part.

It is preferred, according to the invention, that the anti-IL-6 agent is a passive immunotherapy agent, such as an antibody or antibody fragment, or a small molecule, and that the other anti-lL-6 agent is an active immunotherapy agent, such as a polypeptide as defined above, and vice versa.

In this context, it is thus possible, according to the invention, to combine the two approaches of prevention or treatment with the administration of a compound blocking the IL-6 pathway before or after the operation and of active anti-IL-6 immunization, which can also be carried out at the same time as or after the administration of a compound directly active against the IL-6 pathway

The invention will be further explained, in a non-limiting manner, with the aid of the following figures and examples.

### DESCRIPTION OF THE FIGURES

FIG. 1 FIG. 1 plots the force-applied paw withdrawal threshold of a mouse (y-axis, in g force) as a function of time since paw incision (x-axis, in days) for mice injected with PBS (controls, stars), anti-IL-6 antibody (passive immunization, squares), or not incised (controls, triangles).

FIG. 2 FIG. 2 plots the force-applied paw withdrawal threshold of a mouse (y-axis, in g force) as a function of time since paw incision (x-axis, in days) for mice injected with PBS (controls, stars), IL-6-derived peptide (active immunization, squares), or not incised (controls, triangles).

### EXAMPLES

The inventors used a mouse model that measures post-operative pain and is known to be a good model of surgery-related pain (Pogatzki et al. (2003) Anesthesiology 99:1023-7: Cowie et al. (2019) Bio Protoc. 9:e3140). This model is interesting for exploring immediate post-operative pain but also chronic pain as the latter has often been associated with inadequate treatment of the former (Pogatzki-Zahn et al. (2017) Pain Rep. 2:e588).

### Method

Briefly, mice under anesthetic inhalation have a skin and muscle incision of about 5 mm in the sole of the foot of the hind leg (the incision starts in front of the heel towards the toes of the foot). The incision is made in two steps, first the skin and then the flexor digitorum brevis muscle after lifting the latter with a curved forceps. The wound is then sutured (5.0 nylon thread) in two points at the skin level. The sutures are removed on the fourth day.

The pain sensitivity tests performed in mice by the inventors are mechanical push tests at the plantar level just at the heel 3 mm from the tip of the incision. Calibrated von Frey filaments corresponding to a force of 0.07 g, 0.16 g, 0.4 g, 0.6 g, 1 g. 1.4 g, and 2 g (i.e. a force of 0.68 to 19.6 mN) are used. Each filament is applied 5 times for about 1 s, with an interval of 10s between each application, starting with the smallest force, then increasing. A withdrawal of the paw following a pressure is considered as a signal, and when there were least 3 withdrawals on the 5 trials with the same filament, it is considered that there is sensitivity to the considered force. The first filament inducing at least 3 withdrawals out of 5 trials corresponds to the mechanical sensitivity threshold of a mouse. If no filament induces a withdrawal, the sensitivity threshold is considered to be 4 g force (39.2 mN), which corresponds to the filament of size greater than that of 2 g force. Sensitivity is measured every day for 7 days starting from the day after incision.

Groups of 6 mice treated with anti-IL-6, not treated with anti-IL-6, incised or not incised are compared.

Two types of treatment were evaluated in this model of post-operative pain: administration of an anti-IL-6 antibody (example 1) and active immunization against an immunogenic peptide derived from murine IL-6 (example 2).

### Example 1 - Passive Immunization

18 adult male C3H/He mice of 20-30 g were divided into 3 groups:
12 mice to be incised receiving PBS (6 mice, group 1) or anti-IL-6 antibody (6 mice, group 2) by intraperitoneal (ip) injection one day before incision;
6 mice (group 3) receiving PBS in ip injection and which will follow the operative protocol but will not be incised (anesthesia and awakening, without incision). PBS is injected ip in 0.3 mL.

0.5 mg of the polyclonal anti-IL-6 antibody (R&D systems, ref AF406 NA) is dissolved in 1 mL of PBS and 0.3 mL of the mixture is injected by the ip route.

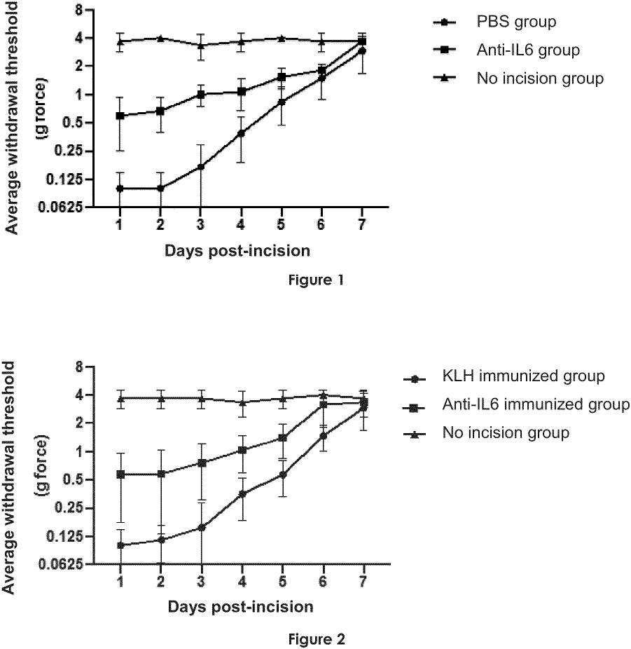

The results of the mechanical sensitivity tests (thresholds obtained) for mice in the 3 groups are summarized in FIG. 1, where the points correspond to the group means with standard deviations:

### Example 2 - Active Immunization

18 adult male C3H/He mice of 20-30 g were divided into 3 groups:
12 mice immunized against KLH carrier protein alone (6 mice, group 1) or against an IL-6-derived peptide coupled to KLH (6 mice, group 2) immunized at D0, D15, D45, and D75 before incision at D80 ;
6 mice immunized against the KLH carrier protein (6 mice, group 3) immunized at D0, D15, D45, and D75 that will follow the surgical protocol at D80 without being incised (anesthesia and awakening without incision).

Immunizations are performed by subcutaneous injection in the upper back of the mouse of 100 µL of ISA51 mixed with 100 µL of KLH protein (amount 80 µg) for groups 1 and 3, and mixed with 100 µL of KLH (amount 80 µg) covalently coupled to the murine IL-6_200 peptide (amount 40 µg) for group 2. The murine IL-6-200 peptide coupled to KLH has the sequence: CMNNDDALAENNLKLPECY (SEQ ID NO: 2) (cyclized by C-C bond) and was previously described in Desallais et al. (2014) Arthritis Research & Therapy 16:R157.

The results of the mechanical sensitivity tests (thresholds obtained) for mice in the 3 groups are summarized in FIG. 2, where the points correspond to the group means with standard deviations.

In both Examples, it can be seen that treatment with an anti-IL-6 antibody, as well as treatment with active anti-IL-6 immunotherapy, lead to significantly less pain sensitivity in the anti-IL-6 treated mice.

1. An anti-IL-6 agent for use in a method of preventing or treating post-operative pain in an individual.

2. The anti-IL-6 agent for use according to claim 1, wherein the anti-IL-6 agent is an immunotherapeutic agent.

3. The anti-IL-6 agent for use claim 1, wherein the anti-IL-6 agent is a passive immunotherapy agent.

4. The anti-IL-6 agent for use claim 1, wherein the anti-IL-6 agent is an anti-IL-6 or anti-IL-6R antibody, in particular monoclonal antibody, or an IL-6 or IL-6R binding portion of an antibody.

5. The anti-IL-6 agent for use claim 1, wherein the anti-IL-6 agent is an active immunotherapy agent.

6. The anti-IL-6 agent for use claim 1, wherein the anti-IL-6 agent comprises, or consists of, a polypeptide derived from IL-6 or the IL-6 receptor (IL-6R), optionally bound to a carrier protein.

7. The anti-IL-6 agent of claim 6, wherein the polypeptide comprises, or consists of, a sequence of at least 6 amino acid residues of IL-6 or IL-6R or a sequence having at least 90% identity thereto.

8. The anti-IL-6 agent for use according to claim 1, wherein the agent is selected from the group consisting of an IL-6 receptor or an IL-6 binding portion thereof or an anti-IL-6 aptamer.

9. The anti-IL-6 agent for use claim 1, wherein post-operative pain is not immediate.

10. The anti-IL-6 agent for use claim 1, wherein the post-operative pain is subsequent to arthroplasty, in particular of the knee or hip, thoracic surgery, in particular cardiac surgery, thoracotomy or sternotomy, breast surgery, hernia surgery, hysterectomy, cholecystectomy, arthroscopy of the knee, or caesarean section.


SEQUENCE LISTING (6 sequences)

SEQ ID NO: 1 - protein, 212 aa, Homo sapiens
MNSFSTSAFGPVAFSLGLLLVLPAAFPAPVPPGEDSKDVAAPHRQPLTSSERIDKQIRYILDGISALRKETCNKSNMCESSKEALAENNLNLPKMAEKDGCFQSGFNEETCLVKIITGLLEFEVYLEYLQNRFESSEEQARAVQMSTKVLIQFLQKKAKNLDAITTPDPTTNASLLTKLQAQNQWLQDMTTHLILRSFKEFLQSSLRALRQM

SEQ ID NO: 2 - protein, 19 aa, Artificial Sequence
  synthetic
CMNNDDALAENNLKLPECY

SEQ ID NO: 3 - protein, 7 aa, Artificial Sequence
  synthetic
ALAENNL

SEQ ID NO: 4 - protein, 468 aa, Homo sapiens
MLAVGCALLAALLAAPGAALAPRRCPAQEVARGVLTSLPGDSVTLTCPGVEPEDNATVHWVLRKPAAGSHPSRWAGMGRRLLLRSVQLHDSGNYSCYRAGRPAGTVHLLVDVPPEEPQLSCFRKSPLSNVVCEWGPRSTPSLTTKAVLLVRKFQNSPAEDFQEPCQYSQESQKFSCQLAVPEGDSSFYIVSMCVASSVGSKFSKTQTFQGCGILQPDPPANITVTAVARNPRWLSVTWQDPHSWNSSFYRLRFELRYRAERSKTFTTWMVKDLQHHCVIHDAWSGLRHVVQLRAQEEFGQGEWSEWSPEAMGTPWTESRSPPAENEVSTPMQALTTNKDDDNILFRDSANATSLPVQDSSSVPLPTFLVAGGSLAFGTLLCIAIVLRFKKTWKLRALKEGKTSMHPPYSLGQLVPERPRPTPVLVPLISPPVSPSSLGSDNTSSHNRPDARDPRSPYDISNTDYFFPR

SEQ ID NO: 5 - protein, 16 aa, Artificial Sequence
  synthetic
ESSKEALAENNLNLPK

SEQ ID NO: 6 - protein, 17 aa, Artificial Sequence
  synthetic
ESSKEALAENNLNLPKC